(19) United States
(12) Patent Application Publication
(10) Pub. No.: US 2006/0293526 A1
(43) Pub. Date: Dec. 28, 2006

(54) Immobilised imidazoles and ruthenium catalysts

(75) Inventors: Katrin Koehler, Gottingen (DE); Dieter Lubda, Bensheim (DE); Kerstin Weigl, Bad Salzungen (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: MERK PATENT GMBH, Darmstadt (DE)

(21) Appl. No.: 10/567,742

(22) PCT Filed: Jul. 16, 2004
(86) PCT No.: PCT/EP04/07939

§ 371 Date: Feb. 10, 2006

(30) Foreign Application Priority Data
Aug. 11, 2003 (DE) 103 37 118.4

(51) Int. Cl. C07F 15/00; C07F 7/02; C07C 2/02; C07C 45/90

(52) U.S. Cl. 548/101; 568/429; 585/527; 556/400

(57) ABSTRACT

The invention relates to the process for the preparation of N,N-disubstituted imidazolium salts, N-heterocyclic carbene ligands and ruthenium catalysts containing N-heterocyclic carbene ligands, i.e. compounds of the general formulae (I) and, (II), compounds of the general formulae (III) and (IV) and compounds of the general formulae (V) and (VI), immobilised on inorganic oxide supports. 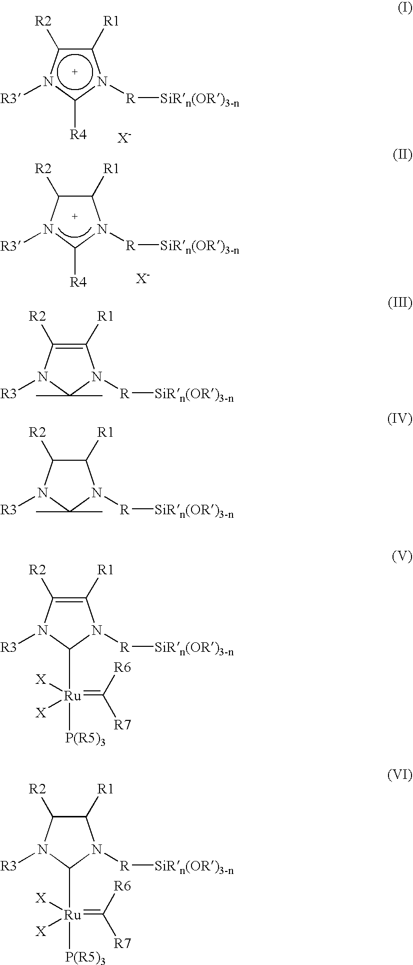 The invention furthermore relates to the use of the immobilised compounds of the general formulae (I-IV) in organic, organometallic or transition metal-catalysed synthesis and to the use of the compounds of the general formulae (V) and (VI) as catalysts in organic and organometallic synthesis, in particular for C—C coupling reactions, such as olefin metathesis.

The invention relates to the process for the preparation of N,N-disubstituted imidazolium salts, N-heterocyclic carbene ligands and ruthenium catalysts containing N-heterocyclic carbene ligands, i.e. compounds of the general formulae (I) and (II), compounds of the general formulae (III) and (IV) and compounds of the general formulae (V) and (VI), immobilised on inorganic oxide supports.

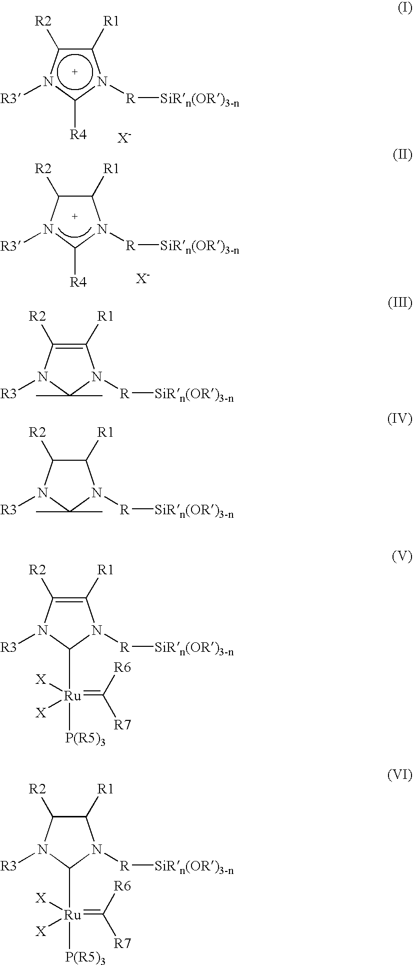

The invention furthermore relates to the use of the immobilised compounds of the general formulae (I-IV) in organic, organometallic or transition metal-catalysed synthesis and to the use of the compounds of the general formulae (V) and (VI) as catalysts in organic and organometallic synthesis, in particular for C—C coupling reactions, such as olefin metathesis.

### 1. Prior Art and Object of the Invention

Examples of sterically undemanding imidazolium and 4,5-dihydroimidazolium salts containing trialkoxysilyl groups are described in WO 01/32308, WO 02/098560 and in J. Am. Chem. Soc. 2002, 124,12932; Topics in Catalysis 2001, 14, 139; Journal of Catalysis 2000, 196, 86; J. Mol. Cat. A: Chem. 2002, 184, 31.

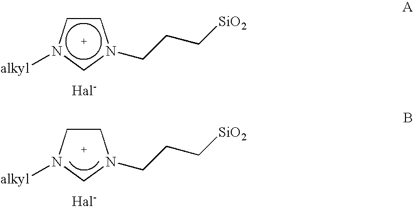

The compounds were either immobilised directly on inorganic oxides or converted into the corresponding surface-modified silica with formation of A and B via a sol-gel method. However, these compounds are unsuitable with respect to use of A and B as ligand precursors for, for example, immobilised NHC (N-heterocyclic carbene) ligands, which are in turn used as ligands in catalysts, since the NHC ligands resulting therefrom are not thermally stable and in addition tend towards dimerisation reactions since the carbene carbon atom is not sterically screened to a sufficient extent. It should be possible to overcome these disadvantages by introducing sterically demanding hydrocarbon radicals, such as substituted aromatic radicals, such as, for example, mesityl radicals, but also adamantyl, cyclohexyl, etc., onto the nitrogen atom in A and B instead of the alkyl group. N-heterocyclic carbene ligands immobilised on inorganic oxides are hitherto unknown.

Examples of ruthenium catalysts containing N-heterocyclic carbene ligands are described, for example, in WO 00/15339, WO 00/71554, WO 99/51344, EP 0721953 and, for example, in Chem. Eur. J. 2001, 7, 3236; J. Am. Chem. Soc. 1999, 121, 2674; Organic Letters 1999, 1(6), 953 and in J. Organomet. Chem. 2000, 606, 49. However, the compounds described can only be employed as homogeneous catalysts. Since the separation of homogeneous catalysts from the reaction products is an expensive and complex procedure, it is of major advantage to employ homogeneous catalysts immobilised on a support in catalytic processes. These immobilised catalysts can be separated off from the reaction products very simply by filtration. This is of major interest, in particular, if the catalyst is very expensive and is thus to be recycled and re-employed in the next catalytic process or if the reaction products of the catalytic process must not be contaminated with transition metals, as are present in the complex compounds. This applies in particular to products for pharmaceutical applications. Immobilisation of ruthenium catalysts containing N-heterocyclic ligands on organic supports, such as polystyrene, is described in Angew. Chem. 2000, 112, 4062. However, organic support materials have many disadvantages compared with very robust inorganic support materials, such as considerable swelling or shrinkage depending on the media used, which can reduce the catalyst activity in an unforeseeable manner. Immobilisation of these catalysts on inorganic oxides has been described by Buchmeiser et al. in Angew. Chem. 2000, 112, 4062, Designed Monomers and Polymers 2002, 5(2,3), 325 and in Adv. Synth. Catal. 2002, 344, 712. The immobilisation method is very complex, and the catalyst is separated from the inorganic oxide by an organic copolymer, i.e. it is ultimately immobilised on an organic support (C).

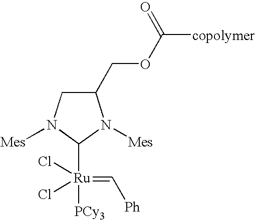

Hoveyda et al. in Angew. Chem. 2001, 113, 4381, report on the immobilisation of a ruthenium catalyst containing an N-heterocyclic carbene ligand on an oxide material with a smaller linker. However, the catalyst is anchored here via the benzylidene ligand. During the catalytic metathesis reaction, however, the bond between the benzylidene ligand and the ruthenium centre is broken, causing the catalyst to be detached from the support and to be transferred into the reaction solution. This results in considerable loss of catalyst on the support (considerable catalyst leaching), which makes reuse with adequate conversions impossible.

The object of the present invention was to immobilise immobilisable, sterically demanding imidazolium and 4,5-dihydroimidazolium salts, immobilisable N-heterocyclic carbene ligands and immobilisable ruthenium catalysts: containing N-heterocyclic carbene ligands on inorganic oxides. At the same time, it should be possible to prepare the immobilised imidazolium and 4,5-dihydroimidazolium salts, the immobilised N-heterocyclic carbene ligands and the immobilised ruthenium catalysts in a simple manner, they should have high thermal stability, they should be covalently bonded to the inorganic support and be available in sufficiently large amount on the support surface for application reactions. They should be strongly anchored to the surface and exhibit no leaching.

### 2. Description of the Invention

The object is achieved by a process for the immobilisation of the compounds of the general formulae (I-VI) 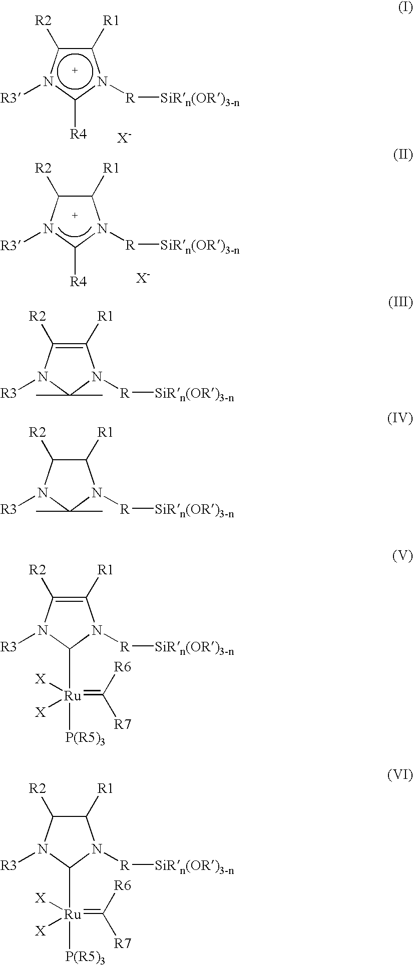
in which
R is A, Ar, A—Ar, A—Ar—A, Het, AHet or AHetA having a total of not more than 30 carbon atoms, where
A is a straight-chain, branched or saturated C₁-C₂₀-alkyl radical, cycloalkyl or cycloalkyl bonded via one or two alkyl group(s) having a total of 4-30 carbon atoms, where one CH₂ or CH group both in the alkyl radical and in the cycloalkyl radical may be replaced by N, NH, NA, O and/or S and H atoms may be replaced by OA, NA₂ and/or PA₂,
Ar is a mono- or polysubstituted or unsubstituted aromatic hydrocarbon having a total of not more than 20 carbon atoms, where substituents may be A, Hal, OA, NA₂, PA₂, COOA, COA, CN, CONHA, NO₂, ═NH or ═O,
Het is a monocyclic or bicyclic, saturated or aromatic heterocyclic radical having from 1 to 4 N, O and/or S atoms, which may be unsubstituted or mono-, di- or trisubstituted by Hal and/or A, OA, COOA, COA, CN, CONHA, NA₂, PA₂, NO₂, ═NH or ═O, where
Hal is F, Cl, Br or I,
R′, independently of the position in the molecule, is A or Ar having 1-12 carbon atoms,
R3 is A, Ar, AAr, AArA, Het, AHet or AHetA having 6-18 carbon atoms,
R3′ is straight-chain or branched cycloalkyl or cycloalkyl bonded via one or two alkyl group(s), Ar, AAr, AArA, Het, AHet or AHetA having a total of 4-30 carbon atoms,
R1 and R2, independently of one another, are H, Cl, Br or are as defined for R3,
R4 is H, Cl, Br or a straight-chain, branched, saturated or mono- or polyunsaturated C₁-C₇-alkyl radical, where one or more H in the alkyl radical may be replaced by Z,
R5 is A, Ar or AAr,
R6 and R7 are H, A or Ar, where H atoms in A or Ar may be substituted by alkenyl or alkynyl radicals,
X are anionic ligands which are identical to or different from one another, and
n is 0, 1 or 2,
   on inorganic oxides with formation of the compounds of the general formulae (Ia-VIa) 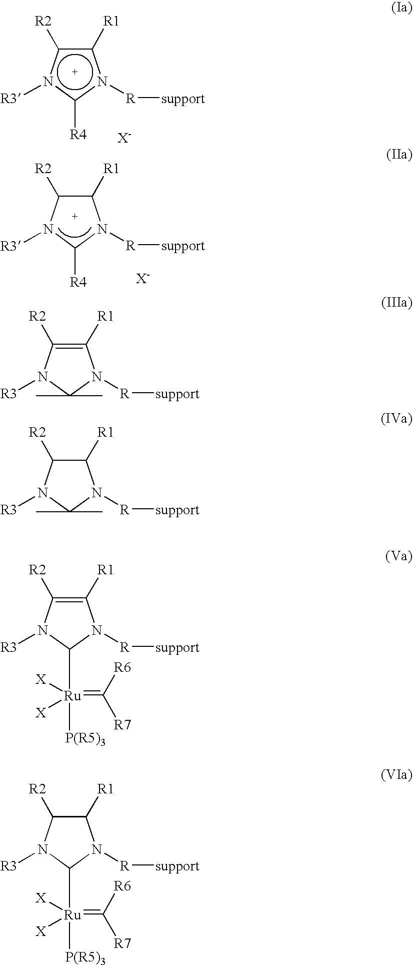
   in which R, R1, R2, R3, R3′, R4, R5, R6, R7 and X can adopt the abovementioned meanings,
   and the corresponding provision of novel support-bound products.

The compounds of the general formulae (I) to (VI) are immobilised by reaction of the compounds (I) to (VI) with an inorganic metal oxide in anhydrous, inert, aprotic, organic solvents. An alcohol R′OH forms as by-product in the reaction. The products (Ia) to (VIa) can be separated off from the solvent and R′OH by filtration and can, if necessary, be purified by washing with a suitable solvent. The immobilisation can be carried out either in a batch process or in a continuous process.

The compounds of the general formulae (Ia) and-(IIa) can be used as immobilised reaction media, immobilised ionic fluids, immobilised ligand or catalyst precursors and as immobilised catalysts in organic, organometallic and transition metal-catalysed syntheses. The compounds of the general formulae (IIIa) and (IVa) can be used as starting materials for the preparation of immobilised N-heterocyclic carbene-metal complexes and as immobilised ligands in catalytic reactions. The compounds of the general formulae (Va) and (VIa) can be used as immobilised catalysts in organic and organometallic synthesis. In particular, they can be used as catalysts in C—C coupling reactions, hydrogenations and hydroformylations.

### 3. DETAILED DESCRIPTION OF THE INVENTION

The compounds of the general formulae (I) to (VI) are immobilised by reaction of the compounds (I) to (VI) with an inorganic metal oxide in anhydrous, inert, aprotic, organic solvents. The sequence of addition of the components can be selected as desired. The starting compounds can be pre-dissolved or suspended in a suitable solvent.

The solvents used are preferably halogenated or pure hydrocarbons and cyclic ethers. Of the halogenated hydrocarbons, preference is given to the use of methylene chloride, chlorobenzene or trichlorotoluene, very preferably methylene chloride. Of the pure hydrocarbons, preference is given to the use of pentane, hexane, heptane, octane, decane, benzene ortoluene, very preferably heptane and toluene. Of the cyclic ethers, preference is given to the use of tetrahydrofuran.

The protective-gas atmosphere used can be nitrogen or argon.

The starting compounds of the general formulae (I) to (VI) are added in a 0.01-1 00-fold excess with respect to the active OH groups on the oxide surface, preferably in a 0.1-50-fold excess, very particularly preferably in a 0.5-1-fold excess.

The reaction can be carried out at a temperature in the range from −20° C. to +150° C., preferably from 0° C. to +120° C. The reaction time is from 30 minutes to 10 days, preferably from 1 hour to 2 days and very preferably from 1 hour to 1 day.

The products (Ia) to (VIa) formed can be separated off in a simple manner by filtration and can, if necessary, be purified by washing with the above-mentioned solvents and subsequently dried.

The immobilisation according to the invention can be carried out either in a batch process or in a continuous process. In the continuous process, the above-described solutions of the compounds (I) to (VI) are pumped through a monolithic or particulate material, during which the corresponding material is warmed to the corresponding reaction temperature. The solutions of (I) to (VI) here can optionally be circulated and thus flow through the monolithic or particulate material a number of times. The flow rates can be selected as desired. The functionalised support is subsequently washed with the above-mentioned solvents and employed in application reactions.

In order to carry out the process according to the invention in batch mode, the oxides can also be mixed in finely divided form with solutions of the compounds of the general formulae (I)-(VI) and reacted at a suitable reaction temperature under a protective-gas atmosphere. For this purpose, the individual reactants can be added in any desired sequence.

The carrying-out of the reactions is not crucial per se either in batch mode or continuously. The reactions can be carried out in a simple manner in plants in which all parts and devices which come into contact with the reactants are inert to the chemicals employed and exhibit no corrosion or leaching phenomena. The crucial factors are that the plant used can be temperature-controlled, offers safe feed and discharge of the reactants and reaction products and, if necessary, has means for intensive mixing of the reaction mixture. The plant should furthermore enable working under an inert-gas atmosphere and safe discharge of volatile substances. Accordingly, the reactions can also be carried out in a glass apparatus equipped with stirrer, feed and optionally discharge; with a reflux condenser or condensation cooler with discharge, if this apparatus also offers the possibility of blanketing with inert gas. However, the reactions can also be carried out in an industrial plant which is manufactured, if appropriate, from stainless steel and other suitable inert materials and has the requisite devices for temperature control, feed and discharge of the starting materials and products.

The reactions are usually carried out in batch mode, in particular if the reactions take place slowly.

If relatively large amounts of the desired products of the general formulae (Ia) to (VIa) are to be produced and if the starting materials to be reacted are reactive compounds, it may be appropriate to carry out the reactions in a corresponding plant which is designed for continuous operation.

Compounds of the general formulae (Ia) and (IIa) according to the invention are immobilised imidazolium and 4,5-dihydroimidazolium salts respectively. (Ia) comprises an immobilised 1,3-disubstituted imidazolium cation with a singly charged anion, and (IIa) comprises an immobilised 1,3-disubstituted 4,5-dihydroimidazolium cation, likewise with a singly charged anion.

Compounds of the general formulae (IIIa) and (IVa) according to the invention are immobilised 1,3-disubstituted imidazol-2-ylidenes and immobilised 1,3-disubstituted imidazolin-2-ylidenes respectively. (IIIa) comprises a 4,5-unsaturated dinitrogen heterocyclic ring, and (IVa) comprises a saturated dinitrogen heterocyclic ring. The carbon atom in the 2-position of the heterocyclic ring (between the two nitrogen atoms) is a divalent carbene carbon atom having a free pair of electrons.

Compounds of the general formulae (Va) and (VIa) according to the invention are immobilised ruthenium compounds in which the ruthenium atom is in oxidation state 2 and to which a neutral N-heterocyclic carbene ligand, a neutral phosphine ligand, a neutral alkylidene ligand and two singly charged anions are bonded. N-heterocyclic carbene ligands are 1,3-disubstituted imidazol-2-ylidenes and 1,3-disubstituted imidazolin-2-ylidenes derived from imidazole or 4,5-dihydroimidazole as parent structures. In both types of ligand, the carbon atom between the two nitrogen atoms of the heterocyclic radical is a carbene carbon atom which is coordinatively bonded to the ruthenium atom by means of the free electron pair. The alkylidene ligand also contains a carbene carbon atom which is bonded to the ruthenium centre.

The compounds (Ia) to (VIa) are bonded to the support surface via a spacer R which corresponds to the hydrocarbon radical R via which the SiR′n(OR′)₃-n group of the compounds of the general formulae (I) to (VI) is bonded to the nitrogen atom of the heterocycle. Accordingly, the spacer R has the same meanings as this hydrocarbon radical.

R′ in the SiR′n(OR′)₃-n unit is a hydrocarbon radical, where n can be 0, 1 or 2, preferably 0 or 1 and very preferably 0. This hydrocarbon radical R′ can adopt different-meanings independently of the position in the molecule and can be straight-chain, unbranched (linear), branched, saturated, mono- or polyunsaturated, cyclic (A), aromatic (Ar) or alkylaromatic (AAr or AArA), and optionally mono- or polysubstituted.

A and Ar can adopt all the meanings given below.

R′ is preferably a straight-chain, unbranched (linear), branched, saturated, mono- or polyunsaturated or cyclic saturated or mono- or polyunsaturated alkyl radical having 1-12 carbon atoms. R′ is particularly preferably a straight-chain or branched saturated alkyl radical having 1-7 carbon atoms, i.e. a sub-group of the alkyl group A, which is defined in greater detail below.

R′ can thus preferably adopt the meanings methyl, ethyl, propyl, i-propyl, butyl, i-butyl, sec-butyl, tert-butyl, pentyl, 1-, 2- or 3-methylbutyl (—C₅H₁₀—), 1,1-, 1,2- or 2,2-dimethylpropyl (—C₅H₁₀—), 1-ethylpropyl (—C₅H₁₀—), hexyl (—C₆H₁₂—), 1-, 2-, 3- or 4-methylpentyl (—C₆H₁₂—), 1,1-, 1,2-, 1,3-, 2,2-, 2,3- or 3,3-dimethylbutyl (—C₆H₁₂—), 1- or 2-ethylbutyl (—C₆H₁₂—), 1-ethyl-1-methylpropyl (—C₆H₁₂—), 1-ethyl-2-methylpropyl (—C₆H₁₂—), 1,1,2- or 1,2,2-trimethylpropyl (—C₆H₁₂—), heptyl, octyl, nonyl, decyl, undecyl or dodecyl.
R′ is very particularly preferably a C₁-C₄-alkyl radical from the group consisting of methyl, ethyl, propyl, i-propyl, butyl i-butyl, sec-butyl and tert-butyl.

In SiR′ₙ(OR′)ₙ₋₃, R′ can, however, alternatively be
alkenyl vinyl, propenyl, 1,2-propadienyl, butenyl, butadienyl, pentenyl, 1,2-, 1,4- or 1,3-pentadienyl, 2,3-dimethyl-2-butenyl, hexenyl, 1,5-hexadienyl, 2-methyl-1,3-butadienyl, 2,3-dimethyl-1,3-butadienyl or isopentenyl,
cycloalkenyl cyclopropenyl, cyclobutenyl, cyclopentenyl, cyclopentadienyl or methylcyclopentadienyl and
alkynyl ethynyl, 1,2-propynyl, 2-butynyl, 1,3-butadiynyl, pentynyl or hexynyl.

The larger the number of alkoxy radicals in the SiR′ₙ(OR′)₃₋ₙ group and thus the smaller is n, the larger can be the number of covalent bonds between the metal oxide and the compounds of the general formulae (I) and (II) after immobilisation. The SiR′ₙ(OR′)₃₋ₙ group is bonded to the nitrogen atom of the heterocyclic radical via a hydrocarbon radical R.

The hydrocarbon radical R is preferably a radical having 1-30 carbon atoms. This hydrocarbon radical may be straight-chain, unbranched (linear), branched, saturated, mono- or polyunsaturated, cyclic (A) or aromatic (Ar), heterocyclic or heteroaromatic (Het) and optionally mono- or polysubstituted.

The hydrocarbon radical R can be an A, Ar, A—Ar, A—Ar—A, Het, A-Het or A-Het-A radical, where each of the groups A, Ar and Het can adopt the meanings given below. R is preferably an A, Ar, A—Ar or A—Ar—A radical having not more than 20 carbon atoms.
A is straight-chain, unbranched (linear), branched, saturated, mono- or polyunsaturated or cyclic alkyl radical A having 1, 2, 3, 4, 5, 6, 7, 8, 9, 10, 11, 12, 13, 14, 15, 16, 17, 18, 19, 20, 21, 22,.23, 24, 25, 26, 27, 28, 29 or 30 carbon atoms, preferably having 1, 2, 3, 4, 5, 6, 7, 8, 9, 10, 11 or 12 carbon atoms.

A is preferably a straight-chain or branched, saturated C₁-C₁₂-alkyl radical or a cycloalkyl having 3-10 carbon atoms or a C₄-C₂₀-cycloalkyl bonded via one or two alkyl group(s).

Alkylene has the same meanings as indicated for A, with the proviso that a further bond exists from the alkyl to the closest bonding neighbour.
A is, for example, an alkylene group selected from the group consisting of methylene (—CH₂—), ethylene (—C₂H₄—), propylene (—C₃H₆—), isopropylene (—C₃H₆—), butylene (—C₄H₈—), isobutylene (—C₄H₈—), sec-butylene (—C₄H₈—) and tert-butylene (—C₄H₈—), furthermore also pentylene (—C₅H₁₀—), 1-, 2- or 3-methylbutylene (—C₅H₁₀—), 1,1-, 1,2- or 2,2-dimethylpropylene (—C₅H₁₀—), 1-ethylpropylene (—C₅H₁₀—), hexylene (—C₆H₁₂—), 1-, 2-, 3- or 4-methylpentylene (—C₆H₁₂—), 1,1-, 1,2-, 1,3-, 2,2-, 2,3- or 3,3-dimethylbutylene (—C₆H₁₂—), 1- or 2-ethylbutylene (—C₆H₁₂-), 1-ethyl-1-methylpropylene (—C₆H12—), 1-ethyl-2-methylpropylene (—C₆H₁₂-), 1,1,2- or 1,2,2-trimethylpropylene (—C₆H₁₂—), heptylene, octylene, nonylene, decylene, undecylene or dodecylene.

A can also be a cycloalkylene group having 3-30 carbon atoms, preferably C₃-C₉-cycloalkylene. Cycloalkyl here can be saturated or unsaturated and optionally bonded via one or two alkyl groups in the molecule to the imidazole nitrogen and the SiR′ₙ(OR′)ₙ₋₃ group. One or more H atom(s) may also be replaced by other substituents in the cycloalkylene group. Cycloalkyl is preferably cyclopropyl, cyclobutyl, cyclopentyl, cyclohexyl, methylcyclopentyl, cycloheptyl, methylcyclohexyl, cyclooctyl, 3-menthyl or camphor-10-yl (bicyclic terpene), decalin or bicycloheptane, where these groups can be bonded via one or two alkyl groups in the molecule to the imidazole nitrogen and the SiR′ₙ(OR′)ₙ₋₃ group. In this case, cycloalkyl is preferably 1,2-cyclopropyl, 1,2- or 1,3-cyclobutyl, 1,2- or 1,3-cyclopentyl, or 1,2-, 1,3- or 1,4-cyclohexyl, furthermore 1,2-, 1,3- or 1,4-cycloheptyl. However, the said groups can also, as R3, be bonded in substituted or unsubstituted form to the second imidazole nitrogen.

A can also be an unsaturated alkenyl or alkynyl group having 2-20 carbon atoms, which can be bonded both to the imidazole nitrogen or an imidazole carbon and to the SiR′ₙ(OR′)ₙ₋₃ group.

Alkenyl groups can be straight-chain, branched or cyclic C₂-C₃₀-alkenyl groups, preferably-straight-chain, branched or cyclic C₂-C₉-alkenyl groups, particularly preferably straight-chain or branched C₂-C₆-alkenyl groups from the group consisting of vinyl, propenyl, butenyl, pentenyl and hexenyl.

Cycloalkenyl groups can be straight-chain or branched C₃-C₃₀-cycloalkenyl groups, preferably C₃-C₉-cycloalkenyl groups, particularly preferably C₃-C₆-cycloalkenyl groups from the group consisting of cyclopropenyl, cyclobutenyl, cyclopentenyl, cyclohexenyl, cyclopentadienyl and methylcyclopentadienyl.

Alkynyl groups can be straight-chain or branched C₂-C₃₀-alkynyl groups, preferably straight-chain or branched C₂-C₉-alkynyl groups, particularly preferably straight-chain or branched C₂-C₆-alkynyl groups from the group consisting of ethynyl, propynyl, butynyl, pentynyl and hexynyl.

If alkenyl, cycloalkenyl or alkynyl is part of the hydrocarbon radical R, it of course has the same meanings, with the proviso that a further bond exists from the alkenyl or from the alkynyl to the closest bonding neighbour in the molecule.

Ar is a mono- or polycyclic aromatic hydrocarbon radical having 6-30 carbon atoms, which may be mono- or polysubstituted or unsubstituted.

Ar is preferably a mono- or polysubstituted phenyl or naphthyl, where substituents can adopt the meanings of A, and Ar has a total of not more than 20 carbon atoms. Aryl groups can preferably be C₆-C₁₀-aryl groups, preferably phenyl or naphthyl. Alkylaryl groups can be C₇-C₁₈-alkylaryl groups, preferably tolyl or mesityl.
Ar is preferably substituted or unsubstituted phenyl, naphthyl, anthryl or phenanthryl, each of which may be mono-, di- or trisubstituted by A, OA, CO—AOH, COOH, COOA, fluorine, chlorine, bromine, iodine, hydroxyl, methoxy, ethoxy, propoxy, butoxy, pentyloxy, hexyloxy, nitro, cyano, formyl, acetyl, propionyl, trifluoromethyl, amino, methylamino, ethylamino, dimethylamino, diethylamino, benzyloxy, sulfoneamido, methylthio, methylsulfinyl, methylsulfonyl, methylsulfonamido, ethylsulfonamido, propylsulfonamido, butylsulfonamido, dimethylsulfonamido, phenylsulfonamido, carboxyl, methoxycarbonyl, ethoxycarbonyl or aminocarbonyl,
where Ar has not more than 20 carbon atoms if it is substituted by A and/or bonded to A.
Ar is preferably unsubstituted or mono- or polysubstituted phenyl, and specifically preferably phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-cyanophenyl, o-, m- or p-methoxyphenyl, o-, m- or p-ethoxyphenyl, o-, m- or p-fluorophenyl, o-, m- or p-bromophenyl, o-, m- or p-chlorophenyl, o-, m- or p-methylthiophenyl, o-, m- or p-methylsulfinylphenyl, o-, m- or p-methylsulfonylphenyl, o-,- m- or p-aminophenyl, o-, m- or p-methylaminophenyl, o-, m- or p-dimethylaminophenyl, o-, m- or p-nitrophenyl, 2,3-, 2,4-, 2,5-, 2,6-, 3,4- or 3,5-difluorophenyl, 2,3-, 2,4-, 2,5-, 2,6-, 3,4- or 3,5-di-chlorophenyl, 2,3-, 2,4-, 2,5-, 2,6-, 3,4- or 3,5-dibromophenyl, 2-chloro-3-methyl-, 2-chloro-4-methyl-, 2-chloro-5-methyl-, 2-chloro-6-methyl-, 2-methyl-3-chloro-, 2-methyl-4-chloro-, 2-methyl-5-chloro-, 2-methyl-6-chloro-, 3-chloro-4-methyl-, 3-chloro-5-methyl- or 3-methyl-4-chlorophenyl, 2-bromo-3-methyl-, 2-bromo-4-methyl-, 2-bromo-5-methyl-, 2-bromo-6-methyl-, 2-methyl-3-bromo-, 2-methyl-4-bromo-, 2-methyl-5-bromo-, 2-methyl-6-bromo-, 3-bromo-4-methyl-, 3-bromo-5-methyl- or 3-methyl-4-bromophenyl, 2,4- or 2,5-dinitrophenyl, 2,5- or 3,4-dimethoxyphenyl, 2,3,4-, 2,3,5-, 2,3,6-, 2,4,6- or 3,4,5-trichlorophenyl, 2,4,6-tri-tert-butylphenyl, 2,5-dimethylphenyl, 4-iodophenyl, 4-fluoro-3-chlorophenyl, 4-fluoro-3,5-dimethylphenyl, 2-fluoro-4-bromophenyl, 2,5-difluoro-4-bromophenyl, 2,4-dichloro-5-methylphenyl, 3-bromo-6-methoxyphenyl, 3-chloro-6-methoxyphenyl, 2-methoxy-5-methylphenyl, 2,4,6-triisopropylphenyl, 1,3-benzodioxol-5-yl, 1,4-benzodioxan-6-yl, benzothiadiazol-5-yl or benzoxadiazol-5-yl or naphthyl.

Arylene has the same meanings as indicated for Ar, with the proviso that a further bond exists from the aromatic system to the closest bonding neighbour.

Specifically, the group referred to as Het can adopt the following meanings:
Het is a mono- or bicyclic saturated, unsaturated or aromatic heterocyclic radical having from 1 to 4 N, O and/or S atoms, which may be unsubstituted or mono-, di- or trisubstituted by Hal and/or A, OA, CO—AOH, COOH, COOA, COA, OH, CN, CONHA, NO₂, ═NH or ═O, where Hal is F, Cl, Br or I.
Het is preferably chromen-2-onyl, pyrrolyl, imidazolyl, pyridyl, pyrimidyl, piperidinyl, 1-methylpiperidinyl, indolyl, thiophenyl, furyl, imidazolyl, pyrazolyl, oxazolyl, isoxazolyl, thiazolyl, isothiazolyl, triazolyl, thienyl, tetrazolyl, oxadiazolyl, thiadiazolyl, thiopyranyl, pyridazinyl, pyrazyl, benzofuryl, benzothienyl, indolyl, 2,1,3-benzothiadiazolyl, benzimidazolyl, benzopyrazolyl, benzoxazolyl, benzisoxazolyl, benzothiazolyl, benzisothiazolyl, benz-2,1,3-oxadiazolyl, quinolyl, isoquinolyl or cinnolinyl, each of which is unsubstituted or mono- or disubstituted by Hal and/or A,
where substituents can be A, OA, CO—AOH, COOH, COOA, fluorine, chlorine, bromine or iodine.
Het is particularly preferably 2- or 3-furyl, 2- or 3-thienyl, 1-, 2- or 3-pyrrolyl, 1-, 2-, 4- or 5-imidazolyl,.1-, 3-, 4- or 5-pyrazolyl, 2-, 4- or 5-oxazolyl, 3-, 4- or 5-isoxazolyl, 2-, 4- or 5-thiazolyl,-3-, 4- or 5-isothiazolyl, 2-, 3- or 4-pyridyl, 1-methylpiperidin-4-yl or piperidin-4-yl, or 2-, 4-, 5- or 6-pyrimidinyl, furthermore preferably 1,2,3-triazol-1-, -4- or -5-yl, 1,2,4-triazol-1-, -3- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 2-, 3-, 4-, 5- or 6-2H-thiopyranyl, 2-, 3- or 4-4-H-thiopyranyl, 3- or 4-pyridazinyl, pyrazinyl, 2-, 3-, 4-, 5-, 6- or 7-benzofuryl, 2-, 3-, 4-, 5-, 6- or 7-benzothienyl, 1-, 2-, 3-, 4-, 5-, 6- or 7-indolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 4- or 5-isoindolyl, 5- or 6-,quinoxalinyl, 2-, 3-, 5-, 6-, 7- or 8-2H-benzo[1,4]oxazinyl, furthermore preferably 1,3-benzodioxol-5-yl, 1,4-benzodioxan-6-yl, 2,1,3-benzothiadiazol-4- or -5-yl, 2,1,3-benzoxadiazol-5-yl or chromenyl.

The heterocyclic radicals may also be partially or completely hydrogenated and adopt the following meanings:
Het is 2,3-dihydro-2-,.-3-, -4- or -5-furyl, 2,5-dihydro-2-, -3-, -4- or -5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-,.-5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8-isoquinolyl, or 2-, 3-, 5-, 6-, 7- or 8-3,4-dihydro-2H-benzo[1,4]oxazinyl, furthermore preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or -6-yl, 2,3-(2-oxomethylenedioxy)phenyl or alternatively 3,4-dihydro-2H-1,5-benzodioxepin-6- or -7-yl, furthermore preferably 2,3-dihydrobenzofuranyl or 2,3-dihydro-2-oxofuranyl.

Heterocycloalkylene or heterocycloarylene has the same meanings as indicated for Het, with the proviso that a further bond exists from the heterocyclic system to the closest bonding neighbour.

Heterocycloalkylene is preferably 1,2-, 2,3- or 1,3-pyrrolidinyl, 1,2-, 2,4-, 4,5- or 1,5-imidazolidinyl, 1,2-, 2,3- or 1,3-pyrazolidinyl, 2,3-, 3,4-, 4,5- or 2,5-oxazolidinyl, 1,2-, 2,3-, 3,4- or 1,4-isoxazolidinyl, 2,3-, 3,4-, 4,5- or 2,5-thiazolidinyl, 2,3-, 3,4-, 4,5- or 2,5-isothiazolidinyl, 1,2-, 2,3-, 3,4- or 1,4-piperidinyl, or 1,4- or 1,2-piperazinyl, furthermore preferably 1,2,3-tetrahydrotriazol-1,2- or -1,4-yl, 1,2,4-tetrahydrotriazol-1,2- or -3,5-yl, 1,2- or 2,5-tetrahydrotetrazolyl, 1,2,3-tetrahydrooxadiazol-2,3-, -3,4-, -4,5- or -1 ;5-yl, 1,2,4-tetrahydrooxadiazol-2,3-, -3,4- or -4,5-yl, 1,3,4-tetrahydrothiadiazol-2,3-, -3,4-, -4,5- or -1,5-yl, 1,2,4-tetrahydrothiadiazol-2,3-, -3,4-, 4,5- or -1,5-yl, 1,2,3-thiadiazol-2,3-, -3,4-, -4,5- or -1,5-yl, 2,3- or 3,4-morpholinyl, or 2,3-, 3,4- or 2,4-thiomorpholinyl.

The hydrocarbon radical R is very particularly preferably a group having not more than 20 carbon atoms and adopts meanings selected from compounds which count amongst the C₁-C₁₂-alkylene groups, C₃-C₁₀-cycloalkylene groups, or C₄-C₂₀-cycloalkylene groups, C₆-C₁₄-arylene groups or C₇-C₂₀-alkylarylene groups, bonded via one or two alkyl group(s), and of these particularly preferably a C₁-C₄-alkylene chain from the series consisting of methylene, ethylene, propylene and butylene or a C₆-C₈-arylene chain from the series consisting of —C₆H₄— and —C₆H₂Me₂— or a C₇-C₉-alkylaryl chain from the series consisting of —CH₂C₆H₄—, —CH₂C₆H₂Me₂—, —CH2C₆H₄CH₂— and —CH₂C₆H₂Me₂CH₂—.

R3 is a hydrocarbon radical which can adopt all meanings of A, Ar, AAr, AArA, Het, AHet or AHetA, in which H atoms may be replaced by functional groups Z. This hydrocarbon radical may be straight-chain, unbranched (linear), branched, saturated, mono- or polyunsaturated, cyclic (A) or aromatic (Ar), heterocyclic or heteroaromatic (Het) and optionally mono- or polysubstituted. The hydrocarbon radical R3 is in particular a radical which exerts a stabilising action on the carbene function of the compounds of the general formulae (I) and (II). The H atoms in R3 may be replaced by functional groups Z as defined below. R3 is preferably an aliphatic, aromatic or heteroaromatic hydrocarbon radical, more precisely, as described above, an aliphatic radical A, an aromatic hydrocarbon Ar from the groups listed above or a heterocyclic substituent Het as defined above. R3 is very preferably an aliphatic, i.e. a straight-chain, unbranched (linear), branched, saturated, mono- or polyunsaturated or cyclic aliphatic or aromatic hydrocarbon radical having 1-18 carbon atoms. From this group of compounds, the radicals phenyl, tolyl, 2,6-dimethylphenyl, mesityl, 2,6-diisopropylphenyl, 2,4,6-triisopropylphenyl or -cyclohexyl have proven particularly suitable and have resulted in particularly advantageous properties of the compounds prepared.

R1 and R2, independently of one another, can be H or can adopt all meanings of Hal, A, Ar and AAr as indicated above, where H atoms in A and Ar may be replaced by functional groups Z, and Hal can be F, Cl, Br or I, R1 and R2 particularly preferably adopt the meanings of R3 or are H, Cl or Br, R1 and R2 are particularly preferably, independently of one another, H, Cl, Br, a straight-chain, branched, saturated or mono- or polyunsaturated C₁-C₇-alkyl radical, where one or more H in the alkyl radical may be replaced by Z.

As already described, H atoms in all hydrocarbon radicals R, R1, R2 and R3, but in particular in R3, may be replaced by functional groups Z and carry N, P, O or S atoms. They can be groups which have one or more alcohol, aldehyde, carboxyl, amine, amide, imide,- phosphine, ether or thioether functions, i.e. they can be, inter alia, radicals having the meanings OA, NHA, NAA′, PAA′, CN, NO₂, SA, SOA, SO₂A or SO₂Ar, where A, A′ and A″, independently of one another, can adopt the meanings of A in accordance with the definition given. They can be groups which have one or more alcohol (OA), aldehyde, carboxyl, amine, amide, imide, phosphine, ether or thioether functions. A group Z preferably has-the meaning OA, NHA, NAA′ or PAA′.

R1 and R2 can therefore, for example, also be SO₃H, F, Cl, or a hydroxyl, alkanoyl or cycloalkanoyl radical.

R1, R2 and R3 can be methoxy, ethoxy, propionyl, butyryl, pentanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl or octadecanoyl.

R1, R2 and R3 can also be acyl radicals. R1, R2 and R3 can preferably be acyl radicals having 1, 2, 3, 4, 5, 6, 7, 8, 9 or 10 carbon atoms and can be, for example, formyl, acetyl, propionyl, butyryl, trifluoroacetyl, benzoyl or naphthoyl. R1, R2 and R3 can furthermore be amino, methylamino, dimethylamino, methylthio, methylsulfinyl, methylsulfonyl or phenylsulfonyl groups.

In addition, one, two or three methylene groups in the radicals R1, R2 and R3 in alkyl, alkylene, cycloalkyl, cycloalkylene, alkanoyl and cycloalkanoyl may each be replaced by N, O and/or S.

A hydrocarbon group in R1, R2 and R3 can thus adopt the meanings of A, Ar or AAr and can be an alkyl, alkenyl, aryl, alkylaryl or alkynyl group as defined above, in which one or more H atoms may be replaced by the above-mentioned functional groups Z.

R3′ is a cyclic hydrocarbon which has a stabilising action on the compounds of the general formulae (I) and (II) compared with the prior art. H atoms in R3′ may be replaced by functional groups Z.

R3′ is preferably a cyclic aliphatic hydrocarbon radical A, as described above, an aromatic hydrocarbon Ar from the groups listed above or a heterocyclic substituent Het as defined above. R3′ is very preferably a cyclic aliphatic or aromatic hydrocarbon radical having 6-18 carbon atoms. From this group of compounds, the radicals mesityl, triisopropylphenyl and cyclohexyl proved particularly suitable and resulted in particularly advantageous properties of the compounds prepared.

In the radicals R3′ and R4, functional groups Z may replace H atoms. These functional groups Z may carry Si, N, P, O or S atoms and can be, inter alia, radicals with the meanings OA, NHA, NAA′, PAA′, CN, NO₂, SA, SOA, SO₂A or SO₂Ar, where A, A′ and A″, independently of one another, can adopt the meanings of A in accordance with the definition given. They can be groups which have one or more alcohol (OA), aldehyde, carboxyl, amine, amide, imide, phosphine, ether or thioether functions. A group Z preferably has the meaning OA, NHA, NAA′ or PAA′.

R4 can therefore also be, for example, SO₃H, F, Cl, a hydroxyl, alkanoyl or cycloalkanoyl radical. They can be methoxy, ethoxy, propionyl, butyryl, pentanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl or octadecanoyl.

R4 can also be an acyl radical. R4 can preferably began acyl radical having 1, 2, 3, 4, 5, 6, 7, 8, 9 or 10 carbon atoms and can be, for example, formyl, acetyl, propionyl, butyryl, trifluoroacetyl, benzoyl or naphthoyl. R1, R2 and R4 can furthermore be amino, methylamino, dimethylamino, methylthio, methylsulfinyl, methylsulfonyl or phenylsulfonyl groups.

It is also possible for one, two or three methylene groups in alkyl, alkylene, cycloalkyl, dycloalkylene, alkanoyl and cycloalkanoyl in the radicals R3′ and R4 each-to be replaced by N, O and/or S.

A hydrocarbon group in R4 can thus adopt the meanings of A, Ar or AAr and can thus be an alkyl, alkenyl, aryl, alkylaryl or alkynyl group as defined above, in which one or more H atoms may be replaced by the above-mentioned functional groups Z.

R4 can be H or can adopt all meanings of Hal, A , Ar and AAr, as indicated above, where H atoms in A and Ar may be replaced by functional groups Z, and Hal can be F, Cl, Br or I. Hal in R4 is preferably Cl or Br. R4 is particularly preferably, independently of one another, H, Cl, Br, or a straight-chain, branched, saturated, mono- or polyunsaturated C₁-C₇-alkyl radical, where one or more H in the alkyl radical may be replaced by Z.

R5 can, independently of one another, be A, Ar or AAr, as defined above, and can in particular be an alkyl, cycloalkyl or aryl group having up to 10 carbon atoms. R5 is preferably C₁-C₆-alkyl, C₅-C₈-cycloalkyl or C₆-C₁₀-aryl and can preferably have the meanings methyl, ethyl, propyl, i-propyl, butyl, i-butyl, sec-butyl, tert-butyl, pentyl, 1-, 2- or 3-methylbutyl (—C₅H₁₀-), 1,1-, 1,2- or 2,2-dimethylpropyl (—C₅H₁₀—), 1-ethylpropyl (—C₅H₁₀—), hexyl (—C₆H₁₂—), 1-, 2-, 3- or 4-methylpentyl (—C₆H₁₂—), 1,1-, 1,2-, 1,3-, 2,2-, 2,3- or 3,3-dimethylbutyl (—C₆H₁₂—), 1- or 2-ethylbutyl (—C₆H₁₂—), 1-ethyl-1 -methylpropyl (—C₆H₁₂—), 1-ethyl-2-methylpropyl (—C₆H₁₂—), 1,1,2- or 1,2,2-trimethylpropyl (—C₆H₁₂—), cyclopentyl, cyclohexyl, methylcyclopentyl, cycloheptyl, methylcyclohexyl, cyclooctyl, phenyl, o-, m- or p-tolyl, o-, m- or p-ethylphenyl, o-, m- or p-propylphenyl, o-, m- or p-isopropylphenyl, o-, m- or p-tert-butylphenyl or naphthyl. R5 is very preferably cyclohexyl, cyclopentyl, isopropyl or phenyl.

R6 and R7, independently of-one another, can be H, A or Ar, where H atoms in A or Ar may be substituted by alkenyl or alkynyl radicals, having not more than 30 carbon atoms. R6 and R7 can therefore, independently of one another, be H, alkyl, cycloalkyl, aryl, alkenyl or alkynyl having up to 30 carbon atoms. R6 and R7 are preferably H, C₁-C₁₀-alkyl, C₆-C₁₀-aryl, C₂-C₁₀-alkenyl or C₂-C₈-alkynyl. R6 and R7 can thus preferably adopt the meanings methyl, ethyl, propyl, i-propyl, butyl, i-butyl, sec-butyl, tert-butyl, pentyl, 1-, 2- or 3-methylbutyl (—C₅H₁₀—) 1, 1 -, 1,2- or 2,2-dimethylpropyl (—C₅H₁₀—), 1-ethylpropyl (—C₅H₁₀—), hexyl (—C₆H₁₂—), 1-, 2-, 3- or 4-methylpentyl (—C₆H₁₂—), 1,1-, 1,2-, 1,3-, 2,2-, 2,3- or 3,3-dimethylbutyl (—C₆H₁₂—), 1- or 2-ethylbutyl (—C₆H₁₂—), 1-ethyl-1-methylpropyl (—C₆H₁₂—), 1-ethyl-2-methylpropyl (—C₆H₁₂—), 1,1 2- or 1,2,2-trimethylpropyl (—C₆H₁₂—), heptyl, octyl, nonyl, decyl, cyclopropenyl, cyclobutenyl, cyclopentenyl, cyclohexenyl, cyclopentadienyl and methylcyclopentadienyl, phenyl, o-, m- or p-tolyl, o-, m- or p-ethylphenyl, o-, m- or p-propylphenyl, o-, m- or p-isopropylphenyl, o-, m- or p-tert-butylphenyl, naphthyl, vinyl, propenyl, butenyl, pentenyl or hexenyl, ethynyl, propynyl, butynyl, pentynyl or hexynyl. R6 and R7 are very preferably H, methyl, phenyl or C₂-C₈-alkenyl, such as, for example, vinyl, —C═CMe₂ or —C═CPh₂.

X is in each case a monovalent anion which serves for charge equalisation. It is bonded in the compounds of the general formulae (V) and (VI) or (Va) and (VIa) as ligand to a doubly positively charged ruthenium central atom. Depending on the electronegativity of the anion X, this bond can be a coordinative bond formed by free electron pairs of the anion, or an ionic bond.

The anions X-present in the compounds (I) and (II) or (V) and (VI) can be, independently of one another, halide (Hal) from the group consisting of Br⁻, Cl⁻, I⁻ and F⁻, pseudohalide, such as cyanide (CN⁻) and thiocyanate (SCN⁻), alkoxide, aryl oxide, alkyl, aryl, carboxyl, etc. X is preferably halide, very preferably Cl or Br.

The compounds of the general formulae (V) and (VI) can basically be prepared by two different methods, which-are referred to below as method A and method B.

The preparation, of the compounds of the general formulae (V) and (VI) can be carried out by method A by reaction of compounds of the general formulae (I) and (II) respectively in accordance-with reaction equations Eq. 1 and Eq. 2 respectively with a base which is capable of deprotonation of (V) and (VI) respectively, such as, for example, metal alkoxides, MOR, metal hydrides, MH, metal amides MNH₂ or ammonia, and [P(R5)₃]₂X₂Ru=CR6R7 in anhydrous, inert, aprotic, organic solvents under a protective-gas atmosphere. After the by-products have been separated off, the compounds of the general formulae (V) and (VI) can be obtained.

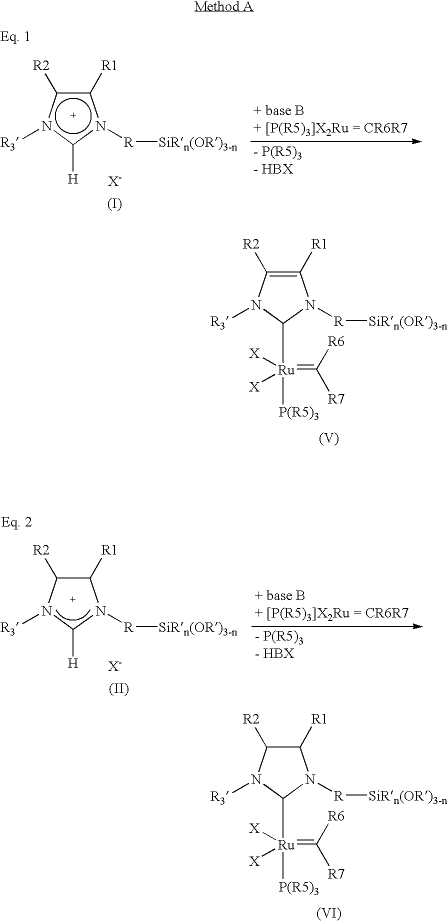

The preparation of the compounds of the general formulae (V) and (VI) can also be carried out by method B by reaction of compounds of the general formulae (Ill) and (IV) respectively analogously to reaction equations Eq. 3 and Eq. 4 respectively with [P(R5)₃]₂X₂Ru=CR6R7 in anhydrous, inert, aprotic, organic solvents. After the by-products have been separated off, the compounds of the general formulae (I) and (II) can be obtained.

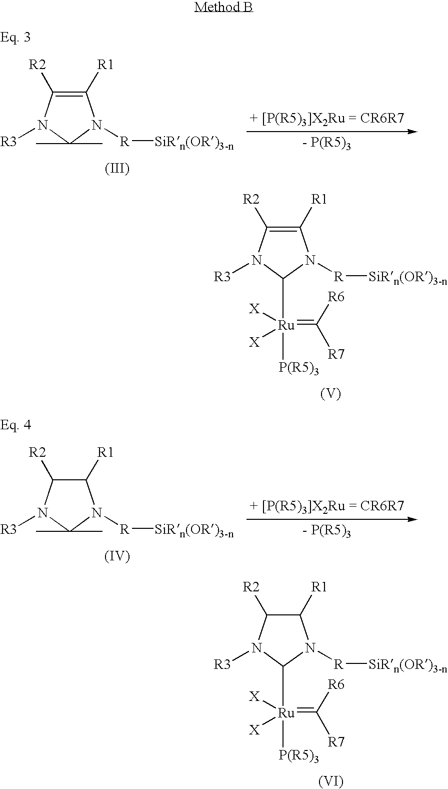

In the case of method B too, the reaction is carried out under a protective-gas atmosphere. Here again, nitrogen and argon are preferred as protective gases. In order to carry out the reaction, the starting materials can be dissolved or suspended in anhydrous, inert, aprotic, organic solvents.

The imidazole parent structures of the substituted imidazoles required as starting materials for the preparation of the compounds of the general formulae (I), (III) and (V) can be prepared analogously to the synthetic method described in Patent Specification U.S. Pat. No. 6,177,575 in accordance with the following general reaction equation:

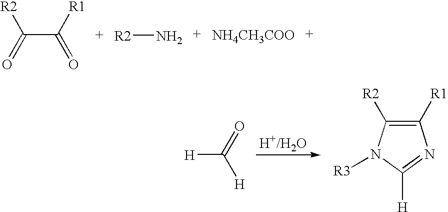

The parent structure of the compounds of the general formulae (II), (IV) and (VI)) (substituted 4,5-dihydroimidazole) can be synthesised by methods which are described in Tetrahedron Lett. 1980, 21, 885, Chem. Ber. 1965, 98, 1342 and in DE-A-11 89 998.

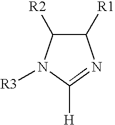

The preparation of the compounds of the general formulae (I) and (II) substituted by silyl groups on the second nitrogen atom of the imidazole ring can be carried out in a simple manner by reaction of an R3-substituted imidazole or substituted 4,5-dihydroimidazole with chlorine-, bromine- or iodine-containing-alkoxysilanes
Hal-R—SiR′ₙ(OR′)₃₋ₙ
without addition of a further solvent under a protective-gas atmosphere.

However, it is also possible to carry out the reaction in an inert, aprotic, organic solvent.

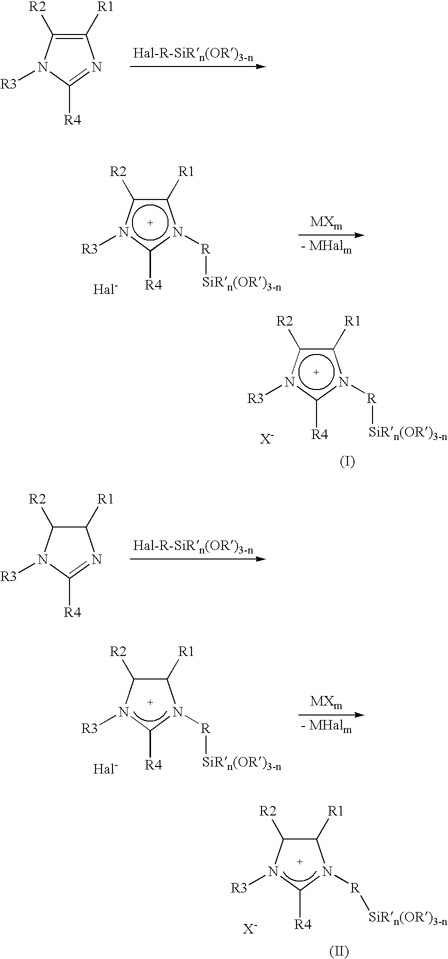

Depending on the reactivity of the imidazole of the general formula employed, the reaction is carried out with maintenance of the reaction temperature within a short time or requires a number of days. The reaction temperature is in the range from 20 to +200° C., preferably from 26 to 100° C. and very preferably between 60 and 100° C. After completion of the reaction, the products (I) and (II) formed can be isolated in pure form as stable substances by known methods and converted further by method A into the compounds of the general formulae (V) and (VI) or immobilised on a support.

The compounds of the general formulae (Ill) and (IV) are prepared by reaction of the alkoxysilyl-functionalised imidazolium salts (I) or alkoxysilyl-functionalised 4,5-dihydroimidazolium salts (II) with a suitable base in anhydrous, inert, aprotic, organic solvents under a protective-gas atmosphere.

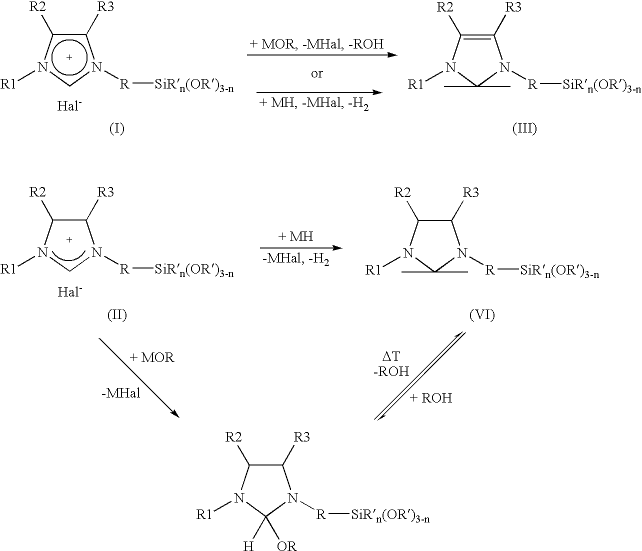

This reaction can, if desired, be carried out directly after the preparation of the imidazolium salts (I) or 4,5-dihydroimidazolium salts (II) without prior purification. Bases which are suitable for this reaction are metal alkoxides of the general formula MOR or bases selected from the group consisting of the metal hydrides MH, metal amides MNH₂ and ammonia in an anhydrous, inert, aprotic,.organic solvent. Preference is given to the use of NH₃/NaH, a metal hydride or a metal alkoxide as base. Potassium t-butoxide (KO^{t}Bu) and potassium hydride (KH) have proven very particularly suitable in various reactions.

For the reaction, all reactants can be introduced together into the reaction vessel. The sequence of addition of the components can be selected as desired.

The starting compounds of the general formulae (I) and (II) can be pre-dissolved or suspended in a suitable solvent, such as, for example, an ether. The protective-gas atmosphere used can be nitrogen or argon. This reaction can be carried out at a temperature in the range from −78° C. to +100° C., preferably from −40° C. to +60° C., for a reaction time of from 1 minute to 6 hours. The products of the general formulae (III) and (IV) formed can, where appropriate after removal of solid by-products and removal of the volatile constituents, be isolated in pure form in a simple manner by extraction and crystallisation or converted directly into the compounds of the general formula (V) or (VI) by method B or immobilised on an inorganic oxide as support.

Supports which can be used are inorganic oxides which contain active OH groups on the surface and are thus capable of reaction with the starting compounds (I) to (VI). Inorganic oxides which can be used are natural or chemically prepared particulate or monolithic oxides of silicon, boron, aluminium, titanium and zirconium or alternatively oxide mixtures. Preference is given to the use of particulate or monolithic oxides of silicon or aluminium or mixed oxides thereof, and zeolites. Particular preference is given to the use of particulate or monolithic oxides of silicon. The silicon-containing materials can be a silica gel or naturally occurring silicate derived from chain-, ribbon- and layer-form silicic acids.

The advantages of the compounds of the general formulae (Ia) and (IIa) compared with, the prior art are that they are very stable due to the sterically demanding radical R3′ and are thus suitable precursors for the synthesis of the thermally very sensitive N-heterocyclic carbene ligands and the metal complexes which can be synthesised therefrom. The advantages of the compounds of the general formulae (IIIa) and (IVa) compared with the prior art are that they are accessible for the first time and that they are also much more thermally stable than their unsupported analogues. Thus, stable, immobilised N-heterocyclic carbene ligands are available for the first time and form with a multiplicity of transition metals very active catalysts which can be employed effectively in organic and organometallic synthesis. The advantages of the compounds of the general formulae (Va) and (VIa) compared with the prior art are that ruthenium catalysts containing an N-heterocyclic carbene ligand which are immobilised directly on an inorganic oxide and are thus very thermally stable are accessible for the first time. The compounds (Ia) to (VIa) are covalently immobilised on the inorganic oxide. They can thus be separated off very simply from the reaction solutions or reaction products in application reactions. The compounds of the general formulae (Ia) to (VIa) can thus be recycled and re-employed in application reactions. This is particularly advantageous in the case of the compounds (Va) and (VIa) since many immobilised catalysts are very expensive and can thus be employed a number of times. This results in a saving of process costs in all application reactions, in particular in catalytic reactions using expensive transition-metal catalysts. Since the SiR′ₙ(OR′)₃₋ₙ group which is capable of immobilisation is bonded to the N-heterocyclic carbene ligand and the latter is bonded more strongly to the ruthenium atom than the P(R5)₃ group still present, immobilised ruthenium catalysts which have no catalyst leaching are accessible for the first time. During the catalytic reaction, the relatively weakly bonded phosphine ligand dissociates from the catalytically active ruthenium centre in the solution, so that the catalytically active species remains bonded to the support throughout the catalysis, and loss of catalyst due to leaching thus does not occur. The compounds of the general formulae (Ia) to (VIa) are accessible very simply and in quantitative yields. In addition, there are inorganic supports which consist either of particles or of a monolith. Consequently, all application reactions can be carried out in batch processes or in continuous processes.

The compounds of the general formulae (Ia) and (IIa) can be used as immobilised reaction media, immobilised ionic fluids, immobilised ligand or catalyst precursors and as immobilised catalysts in organic, organometallic and transition metal-catalysed syntheses. The compounds of the general formulae (IIIa) and (IVa) can be used as starting materials for the preparation of immobilised N-heterocyclic carbene-metal complexes and as immobilised ligands in catalytic reactions, in particular in ruthenium-catalysed metathesis reactions, palladium-catalysed Heck or Suzuki reactions, rhodium-catalysed hydrogenations; furan syntheses, hydroformylations, isomerisations or hydrosilylations. The compounds of the general formulae (Va) and (VIa) can be used as immobilised catalysts in organic and organo-metallic synthesis. In particular, they can bemused as catalysts in C—C coupling reactions, hydrogenations, isomerisations, silylations and hydroformylation. The novel compounds are particularly suitable as immobilised catalysts for C—C coupling reactions, such as olefin metathesis, and for hydrogenation reactions. The novel compounds are particularly advantageous in olefin metathesis reactions, such as cross metathesis (CM), ring closure metathesis (RCM), ring opening metathesis polymerisation (ROMP), acyclic diene metathesis polymerisation (ADMET) and ene-yne metathesis.

### 4. EXAMPLES

For better understanding and in order to clarify the invention, examples are given below which are within the scope of protection of the present invention. However, owing to the general validity of the inventive principle described, these are not suitable for reducing the scope of protection of the present application merely to these examples.

(A) Immobilisation of N,N′-disubstituted Imidazolium Salts

### Immobilisation of 1-mesityl-3-[3-(triethoxysilyl)propyl]imidazolium chloride on silica gel 60 in CH₂Cl₂

2.64 g of silica gel 60 and a solution of 2.68 g (6.3 mmol) of 1-mesityl-3-[3-(triethoxysilyl)propyl]imidazolium chloride and 50 ml of CH₂Cl₂ are introduced under an argon atmosphere into a flask fitted with reflux condenser. The mixture is refluxed overnight, and the silica gel is subsequently filtered off and washed with CH₂Cl₂ until the washings remain colourless. The solid is dried under reduced pressure, giving the product as a pale-brown powder.

Anaysis [%]: found: C 11.8, H 1.7, N 1.4. Loading [μmol/m²]: C 1.62, N 1.17. ¹³C MAS NMR: δ 0-34 (Cₐₗᵢ), 44-64 (NC, OC), 116-146 (Cₐᵣₒₘ). ²⁹Si MAS-NMR: δ −107 (Q₃, 25%), −98 (Q₂, 44%), −89 (Q₁, 7%), −65 (T₄, 10%), −58 (T₃, 3%), −51 (T′₁, 2%), −50 (T₁, 9%).

### Immobilisation of 1-mesityl-3-[3-(triethoxysilyl)propyl]imidazolium chloride on silica gel 60 in toluene

3.07 g of silica gel 60 and a solution of 5.38 g (12.6 mmol) of 1-mesityl-3-[3-(triethoxysilyl)propyl]imidazolium chloride and 50 ml of toluene are introduced under an argon atmosphere into a flask fitted with reflux condenser. The mixture is refluxed overnight. The silica gel is filtered off and washed with CH₂Cl₂ until the washings remain colourless and subsequently dried under reduced pressure, giving the product as a pale-brown powder.

Analysis [%]: found: C 12.3, H 2.2, N 1.4. Loading [μmol/²]: C. 1.72, N 1.27. ¹³C MAS NMR: δ 4-32 (Cₐₗᵢ), 46-64 (NC, OC), 116-146 (Cₐᵣₒₘ). ²⁹Si MAS NMR: δ −108 (Q₃, 34%), −98 (Q₂, 43%), −89 (Q₁, 5%), −49 (T′₁, 18%).

### Immobilisation of 1-mesityl-3-[4-(trimethoxysilyl)benzyl]imidazolium chloride on silica gel 60 in CH₂Cl₂

3.00 g (6.94 mmol) of 1-mesityl-3-[4-(trimethoxysilyl)benzyl]imidazolium chloride, 2.69 g of silica gel 60 and 25 ml of CH₂Cl₂ are introduced under an argon atmosphere into a nitrogen flask. The mixture is refluxed over-night. The silica gel is separated off from the solution and washed three times with CH₂Cl₂ and subsequently dried under reduced pressure, giving the product as a brown powder.

Analysis [%]: found: C 12.5, H 3.0, N 1.3. Loading [μmol/m²]: C 1.35, N 1.10. ¹³C MAS NMR: δ ?12 -26 (Caₐₗᵢ), 44-56 (NC, OC), 146-120 (Cₐᵣₒₘ). ²⁹Si MAS NMR: δ −108 (Q₃, 39%), −98 (Q₂, 49%), −89 (Q₁, 3%), −68 (T₄, 1%), −60 (T₃, 5%), −53 (T₁′, 2%), −49 (T₁, 1%).

### Immobilisation of 1-mesityl-3-[4-(trimethoxysilyl)benzyl]imidazolium chloride on silica gel 60 in toluene

3.00 g (6.94 mmol) of 1-mesityl-3-[4-(trimethoxysilyl)benzyl]imidazolium chloride, 2.71 g of silica gel 60 and 25 ml of toluene are introduced under an argon atmosphere into a nitrogen flask. The mixture is refluxed over-night. The silica gel is separated off from the solution and washed three times with CH₂Cl₂ and subsequently dried under reduced pressure, giving the product as a brown powder.

Analysis [%]: found:.C 16.7, H 2.6, N 1.6. Loading [μmol/m²]: C 1.95, N 1.42. ¹³C MAS NMR:? δ 12-26 (Cₐₗᵢ), 44-58 (NC, OC), 150-120 (Cₐᵣₒₘ).

### Immobilisation of 1-mesityl-3-[3-(triethoxysilyl)propyl]imidazolium chloride on a silica monolith in the flask

624 mg (1.46 mmol) of 1-mesityl-3-[3-(triethoxysilyl)propyl]imidazolium chloride are dissolved in 5 ml of CH₂Cl₂ in a nitrogen flask with attached reflux condenser. The monolith is placed in this solution and is allowed to suck up the liquid slowly until full. The mixture is refluxed overnight. The solution is removed. The monolith is washed with CH₂Cl₂ until the washings remain colourless, giving the product as a pale-brown monolith rod.

Analysis [%]:found: 12.4% C, 2.8% H, 1.5% N. Loading [μmol/m²]: C 1.72, N 1.27. ¹³C MAS NMR: δ ?8-36 (Cₐₗᵢ), 52-62 (NC, OC), 146-120, (Cₐᵣₒₘ). ²⁹Si MAS NMR: δ T₁′ (7%), T₄ (4%), Q₁ (1%), Q₂ (43%), Q₃ (46%).

### Immobilisation of 1-mesityl-3-[3-(triethoxysilyl)propyl]imidazolium chloride on a chromolith in through-flow

The monolith pre-dried overnight at 80° C. in a drying cabinet is installed in the through-flow apparatus oven set to a temperature of 30° C. It is rinsed with CH₂Cl₂ for 1 hour at a flow rate of 0.05 ml/min. 1.03 g (2.00 mmol) of 1-mesityl-3-[3-(triethoxysilyl)propyl]imidazolium chloride dissolved in 50 ml of CH₂Cl₂ are introduced in 10 ml portions into the sample loop and pumped through the monolith at a flow rate of 0.3 ml/mm. Rinsing was carried out overnight with CH₂Cl₂ at a flow rate of 0.1 ml/min.

Analysis [%]: found: 10.6% C, 1.7% H, 1.3% N. Loading [μmol/m²]: C 1.42, N 1.17. ¹³C MAS NMR: δ 4-32 (Cₐₗᵢ), 46-60 (NC, OC), 146-116 (Cₐᵣₒₘ). ²⁹Si MAS NMR: δ T₃ (7%), T₄ (11%). Q₁ (5%), Q₂ (46%), Q₃ (31%).

(B) Immobilisation of N-heterocyclic Carbenes

### Immobilisation of 1-[3-(trimethoxysilyl)benzyl]-3-(mesityl)imidazol-2-ylidene on silica gel 60

1.5 g (3.77 mmol) of 1-[4-(trimethoxysilyl)benzyl]-3-(mesityl)imidazolium chloride, 403 mg (3.60 mmol) of KO^{t}Bu and 15 ml of THF are introduced under an argon atmosphere into a Schlenk tube and stirred at RT for 1 hour. The volatile constituents are removed under reduced pressure. The residue is taken up in 25 ml of heptane. The solution is separated from the resultant solid by filtration and transferred via a cannula into a second Schlenk tube into which 1.44 g of silica gel 60 have been introduced. The mixture is stirred at RT for 3 hours. The silica gel is subsequently separated off via a frit, washed with heptane and dried in a high vacuum, giving the product as a free-flowing powder.

Analysis [%] found: C 17.7, H 2.4, N 1.7. Loading [μmol/m²]: C 2.03, N 1.49.

(C) Immobilisation of Ruthenium Catalysts

### Immobilisation of {1-mesityl-3-[3-(triethoxysilyl)propyl]imidazol-2-ylidene}(PCy₃)Cl₂Ru=CHPh on silica gel 60 in heptane

380 mg of {1-mesityl-3-[3-(triethoxysilyl)propyl]imidazol-2-ylidene}-(PCy₃)Cl₂Ru=CHPh are dissolved in 15 ml of CH₂Cl₂ under an argon atmosphere, and 100 mg of silica gel 60 are added. The mixture is stirred at 25° C. for 18 hours. The silica gel 60 is separated off from the solution by filtration and repeated washing with heptane and tetrahydrofuran. The functionalised silica gel is dried in a high vacuum.

Analysis [%]: found: C 14.0, H 2.3, N 1.0. Loading [μmol/m²]: C 0.8, N 0.8. ¹³C MAS NMR: δ 10-30 (Cₐₗᵢ), 132 -120 (Cₐᵣₒₘ).

### Immobilisation of {1-mesityl-3-[3-(triethoxysilyl)propyl]imidazol-2-ylidene}(PCy₃)Cl₂Ru=CHPh on an SiO₂ monolith in through-flow

The SiO₂ monolith is dried overnight at 120° C. in a drying cabinet and subsequently connected in the thermostat of the through-flow apparatus. The chromolith is rinsed with CH₂Cl₂ for 1 hour at a flow rate of 0.5 ml/min. 950 mg of {1-mesityl-3-[3-(triethoxysilyl)propyl]imidazol-2-ylidene}-(PCy₃)Cl₂Ru=CHPh) are dissolved in 30 ml of CH₂Cl₂ under an argon atmosphere in a flask and injected into the sample loop of the through-flow apparatus. The immobilisation is carried out at a flow rate of 0.03 ml/min. Rinsing is subsequently carried out with 30 ml of tetrahydrofuran and with 20 ml of CH₂Cl₂.

### Immobilisation of {1-mesityl-3-[4-(trimethoxysilyl)benzyl]imidazol-2-ylidene}(PCy₃)C₁₂Ru=CHPh on silica gel 60 in methylene chloride

212 mg of {1-mesityl-3-[4-(trimethoxysilyl)benzyl]imidazol-2-ylidene}-(PCy₃)Cl₂Ru=CHPh are dissolved in 5 ml of dichloromethane under an argon atmosphere. This solution is added to 70 mg of silica gel 60 and refluxed for 24 hours. The supernatant solution is separated off using a cannula, and the precipitate is washed three times with heptane, three times with THF and once with dichloromethane. Drying in a high vacuum gives a brown powder.

Analysis [%]: found: C 12.2, H 2.1, N 0.5. Loading [μmol/m²]: C 0.57, N 0.42.

(D) Testing of the Immobilised Ruthenium Catalysts in Catalysis

### Metathesis using {1-mesityl-3-[3-(triethoxysilyl)propyl]imidazol-2-ylidene}(PCy₃)Cl₂Ru=CHPh immobilised on silica gel 60

40 μmol of {1-mesityl-3-[3-(triethoxysilyl)propyl]imidazol-2-ylidene}-(PCy₃)Cl₂Ru=CHPh) immobilised on silica gel 60. (100 mg), 86.4 ml (4 mmol) of 1,7-octadiene and 50 ml of CH₂Cl₂ are introduced under an argon atmosphere into a three-necked flask. The mixture is stirred under reflux, and samples are taken for gas chromatography. GC: 1,7-octadiene: cyclohexene ratio: 1:4.4 (81 % conversion).

### Catalyst leaching test with {1-mesityl-3-[3-(triethoxysilyl)propyl]imidazol-2-ylidene}(PCy₃)Cl₂Ru=CHPh immobilised on silica gel 60

40 μmol of {1-mesityl-3-[3-(triethoxysilyl)propyl]imidazol-2-ylidene}-(PCy₃)C₁₂Ru=CHPh immobilised on silica gel 60 (100 mg) and 50 ml of CH₂Cl₂ are introduced under an argon atmosphere into a three-necked flask. The catalyst is separated off, and 86.4 ml (4 mmol) of 1,7-octadiene are added to the solution. The mixture is stirred under reflux, and samples are taken for gas chromatography. No cyclohexene could be detected in the GC. The immobilised catalyst has thus not been transferred into the solution.

### Metathesis using {1-mesityl-3-[3-(triethoxysilyl)propyl]imidazol-2-ylidene}(PCy₃)Cl₂Ru=CHPh immobilised on an SiO₂ monolith

The SiO₂ monolith functionalised with {1-mesityl-3-[3-(triethoxysilyl)propyl]-imidazol-2-ylidene}(PCy₃)Cl₂Ru=CHPh is installed in the through-flow apparatus. 8 ml (53 mmol) of 1,7-octadiene are introduced into the sample loop and pumped over the chromolith once at a flow rate of 0.5 ml/min at room temperature. The reaction solution obtained is analysed by GC. GC: 1,7-octadiene:cyclohexene ratio: 1.00:1.67 (62%).

1. Process for the immobilisation of N,N-disubstituted imidazolium salts, N-heterocyclic carbene ligands and ruthenium catalysts containing N-heterocyclic carbene ligands on inorganic oxide supports, characterised in that a compound of the general formula (I), (II), (III), (IV), (V) or (VI) 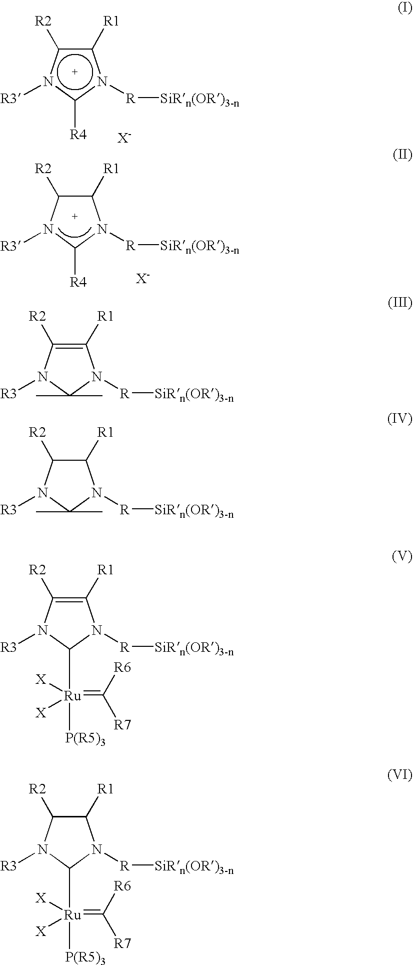
in which
R is A, Ar, A—Ar, A—Ar—A, Het, AHet or AHetA having a total of not more than 30 carbon atoms, where
A is a straight-chain, branched or saturated C₁-C₂₀-alkyl radical, cycloalkyl or cycloalkyl bonded via one or two alkyl group(s) having a total of 4-30 carbon atoms, where one CH₂ or CH group both in the alkyl radical and in the cycloalkyl radical may be replaced by N, NH, NA, O and/or S and H atoms may be replaced by OA, NA₂ and/or PA₂,
Ar is a mono- or polysubstituted or unsubstituted aromatic hydrocarbon having a total of not more than 20 carbon atoms, where substituents may be A, Hal, OA, NA₂, PA₂, COOA, COA, CN, CONHA, NO₂, ═NH or ═O,
Het is a monocyclic or bicyclic, saturated or aromatic heterocyclic radical having from 1 to 4 N, O and/or S atoms, which may be unsubstituted or mono-, di- or trisubstituted by Hal and/or A, OA, COOA, COA, CN, CONHA, NA₂, PA₂, NO₂, ═NH or ═O, where
Hal is F, Cl, Br or I,
R′, independently of the position in the molecule, is A or Ar having 1-12 carbon atoms,
R3 is A, Ar, AAr, AArA, Het, AHet or AHetA having 6-18 carbon atoms,
R3′ is straight-chain or branched cycloalkyl or cycloalkyl bonded via one or two alkyl group(s), Ar, AAr, AArA, Het, AHet or AHetA having a total of 4-30 carbon atoms,
R1 and R2, independently of one another, are H, Cl, Br or are as defined for R3,
R4 is H, Cl, Br or a straight-chain, branched, saturated or mono- or polyunsaturated C₁-C₇-alkyl radical, where one or more H in the alkyl radical may be replaced by Z,
R5 is A, Ar or AAr,
R6 and R7 are H, A or Ar, where H atoms in A or Ar may be substituted by alkenyl or alkynyl radicals,
X are anionic ligands which are identical to or different from one another, and
n is 0, 1 or 2,
is reacted with an inorganic metal oxide containing active OH groups on the surface, in an anhydrous, inert, aprotic, organic solvent with formation of an alcohol R′OH under a protective-gas atmosphere, the product (Ia), (IIa), (IIa), (IVa), (Va) or (VIa) 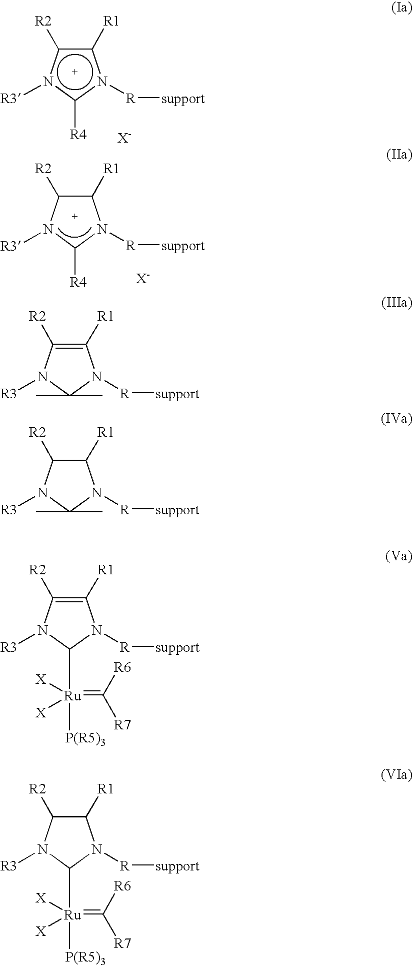
in which R, R1, R2, R3, R3′, R4, R5, R6, R7 and X are as defined above, and “-support” denotes an inorganic oxide, is separated off and, if necessary, purified.

2. Process according to claim 1, characterised in that the protective-gas atmosphere used is nitrogen or argon.

3. Process according to claim 1, characterised in that the product formed is separated off by filtration and, if necessary, purified by washing with a suitable solvent.

4. Process according to claim 1, characterised in that it is carried out by the batch method.

5. Process according to claim 1, characterised in that it is carried out by a continuous method.

6. Process according to claim 1, characterised in that the inorganic oxides used are natural or chemically prepared particulate or monolithic oxides of silicon, boron, aluminium, titanium and zirconium or mixed oxides thereof, or zeolites.

7. Process according to claim 1, characterised in that the inorganic oxides used are particulate or monolithic oxides of silicon or aluminium or mixed oxides thereof.

8. Process according to claim 1, characterised in that the inorganic oxides used are particulate or monolithic oxides of silicon, which can be a silica gel or naturally occurring silicate derived from chain-, ribbon- and layer-form silicic acids.

9. Process according to claim 1, characterised in that the solvents used are hydrocarbons, halogenated hydrocarbons or cyclic ethers.

10. Process according to claim 1, characterised in that the solvents used are pentane, hexane, heptane, octane, decane, benzene, toluene, methylene chloride, chlorobenzene, trichlorotoluene, tetrahydrofuran or mixtures thereof.

11. Process according to claim 1, characterised in that the starting compounds of the general formulae (I) to (VI) are added in a 0.01-100-fold excess with respect to the active OH groups on the oxide surface, preferably in a 0.1-50-fold excess and very particularly preferably in a 0.5-10-fold excess.

12. Process according to claim 1, characterised in that the reaction is carried out at a temperature in a range between −20° C. and +150° C., preferably between 0° C. and +120° C., within a reaction time of from 30 minutes to 10 days, preferably from one hour to 2 days and very preferably from one hour to one day.

13. Process according to claim 1, characterised in that, when the reaction is complete, the products (Ia) to (VIa) formed are separated off by filtration, optionally washed with a solvent selected from the group consisting of pentane, hexane, heptane, octane, decane, benzene, toluene, methylene chloride, chlorobenzene, trichlorotoluene and tetrahydrofuran or mixtures thereof, and subsequently dried.

14. Process according to claim 1, characterised in that solutions of the compounds of the general formulae (I) to (VI) are pumped continuously through the monolithic material, where the monolith is set to a temperature between −20° C. and +150° C., and the functionalised monolith is washed, when the reaction is complete, with a solvent selected from the group consisting of pentane, hexane, heptane, octane, decane, benzene, toluene, methylene chloride, chlorobenzene, trichlorotoluene and tetrahydrofuran or mixtures thereof.

15. Process according to claim 14, characterised in that solutions of the compounds of the general formulae (I) to (VI) are circulated continuously by pump, causing repeated flow through the monolith.

16. Process according to claim 1, characterised in that the inorganic oxides are mixed and brought to reaction with the solutions of the compounds of the general formulae (I) to (VI).

17. N,N-disubstituted imidazolium salts of the general formulae (Ia) and (IIa), N-heterocyclic carbene ligands of the general formulae (IIIa) and (IVa) and ruthenium catalysts containing N-heterocyclic carbene ligands of the general formulae (Va) and (VIa) immobilised on inorganic oxide supports.

18. Use of the compounds of the general formulae (Ia) and (IIa) as immobilised reaction media, immobilised ionic fluids, immobilised ligand or catalyst precursors and as immobilised catalysts in organic, organometallic and transition metal-catalysed syntheses.

19. Use of the compounds of the general formulae (IIIa) and (IVa) as starting materials for the preparation of immobilised N-heterocyclic carbene-metal complexes.

20. Use of the compounds of the general formulae (IIIa) and (IVa) as immobilised ligands in catalytic reactions, in particular in ruthenium-catalysed metathesis reactions, palladium-catalysed Heck or Suzuki reactions, rhodium-catalysed hydrogenations, furan syntheses, hydroformylations, isomerisations or hydrosilylations.

21. Use of the compounds of the general formulae (Va) and (VIa) as immobilised catalysts in organic and organometallic synthesis.

22. Use of the compounds of the general formulae (Va) and (VIa) as catalysts in C—C coupling reactions, hydrogenations, isomerisations, silylations and hydroformylations.

23. Use of the compounds of the general formulae (Va) and (VIa) as immobilised catalysts for C—C coupling reactions, such as olefin metathesis, and for hydrogenation reactions, olefin metathesis reactions, such as cross metathesis (CM), ring closure metathesis (RCM), ring opening metathesis polymerisation (ROMP), acyclic diene metathesis polymerisation (ADMET) and ene-yne metathesis.
